(19) United States
(12) Patent Application Publication
(10) Pub. No.: US 2009/0156828 A1
(43) Pub. Date: Jun. 18, 2009

(54) Novel Compounds and Methods for Forming Taxanes and Using the Same

(75) Inventors: John T. Henri, Longmont, CO (US); James D. McChesney, Boulder, CO (US); Sylesh Venkataraman, Longmont, CO (US); Christian Sumner, Boulder, CO (US); George Petros Yiannikouros, Florence, SC (US); Aaron Michael Stemphoski, Florence, SC (US); Donald G. Walker, Florence, SC (US)

Correspondence Address: MORGAN, LEWIS & BOCKIUS LLP, 1701 MARKET STREET, PHILADELPHIA, PA, 19103-2921, US

(73) Assignee: Tapestry Pharmaceuticals, Inc.

(21) Appl. No.: 12/097,900

(22) PCT Filed: Dec. 21, 2005
(86) PCT No.: PCT/US05/46887

§ 371 Date: Dec. 1, 2008

(51) Int. Cl. C07D 263/02; C07D 305/14

(52) U.S. Cl. 548/215; 549/510

(57) ABSTRACT

The present invention is broadly directed to novel compounds useful for the synthesis of biologically active compounds. More particularly, the present embodiments disclosed herein relate to novel side chains, that when coupled to a taxane, are useful for the synthesis of pharmaceutically useful taxanes. Methods of forming the novel side chains and coupling them to hindered alcohols, namely taxanes resulting in useful esters are also disclosed. Various taxanes compounds are known to exhibit anti-tumor activity.

### CROSS-REFERENCE TO RELATED APPLICATIONS

This application is a Continuation-in-Part of U.S. patent application Ser. No. 10/951,555, filed Sep. 27, 2004 and PCT Application No. PCT/US04/31816 filed Sep. 27, 2004, both of which are currently pending.

### FIELD OF THE INVENTION

The present invention is broadly directed to novel compounds useful for the synthesis of biologically active compounds. More particularly, the present embodiments disclosed herein relate to novel side chains, that when coupled to a taxane, are useful for the synthesis of pharmaceutically useful taxanes. Methods of forming the novel side chains and coupling them to hindered alcohols, namely taxanes resulting in useful esters are also disclosed.

### BACKGROUND OF THE INVENTION

Various taxane compounds are known to exhibit anti-tumor activity. As a result of this activity, taxanes have received increasing attention in the scientific and medical community, and are considered to be an exceptionally promising family of cancer chemotherapeutic agents. For example, taxanes such as paclitaxel and docetaxel have been approved for the chemotherapeutic treatment of several different varieties of tumors. As is known, paclitaxel is a naturally occurring taxane diterpenoid having the formula and numbering system for the taxane backbone as follows:

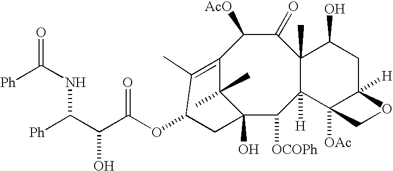

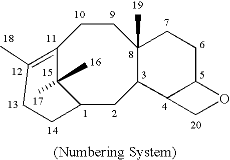

Since the paclitaxel compound appears so promising as a chemotherapeutic agent, organic chemists have spent substantial time and resources in attempting to synthesize the paclitaxel molecule and other potent taxane analogs. The straightforward implementation of partial synthesis of paclitaxel, or other taxanes, requires convenient access to chiral, non-racemic side chains and derivatives, an abundant natural source of baccatin III or closely related diterpenoid substances, and an effective means of joining the two. Perhaps the most direct synthesis of paclitaxel is the condensation of Baccatin III and 10-deacetylbaccatin III of the formulae:

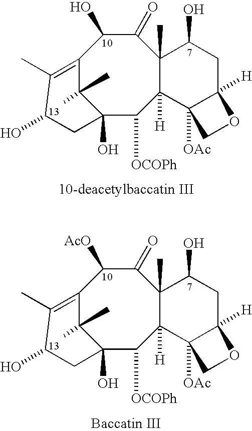

with the side chain:

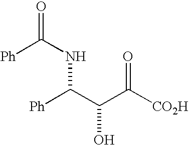

However, the esterification of these two units is difficult because of the C-13 hydroxyl of both baccatin III and 10-deacetylbaccatin III are located within the sterically encumbered concave region of the hemispherical taxane skeleton.

Alternative methods of coupling the side chain to a taxane backbone to ultimately produce paclitaxel have been disclosed in various patents. For example, U.S. Pat. No. 4,929,011 issued May 8, 1990 to Denis et al. entitled “Process for Preparing Taxol”, describes the semi-synthesis of paclitaxel from the condensation of a (2R,3S) side chain acid of the general formula:

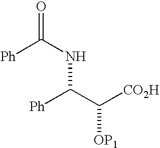

wherein P₁ is a hydroxyl protecting group with a taxane derivative of the general formula of:

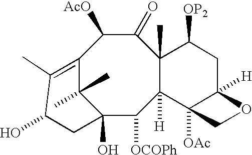

wherein P₂ is a hydroxyl protecting group. The condensation product is subsequently processed to remove the P₁ and P₂ protecting groups. In Denis et al., the paclitaxel C-13 side chain, (2R,3S) 3-phenylisoserine derivative is protected with P₁ for coupling with protected Baccatin III. The P₂ protecting group on the baccatin III backbone is, for example, a trimethylsilyl or a trialkylsilyl radical.

An alternative semi-synthesis of paclitaxel is described in U.S. Pat. No. 5,770,745 to Swindell et al. Swindell et al. disclose semi-synthesis of paclitaxel from a baccatin III backbone by the condensation with a side chain having the general formula:

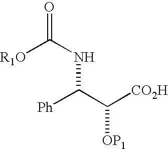

wherein R₁ is alkyl, olefinic or aromatic or PhCH₂ and P₁ is a hydroxyl protecting group.

Another technique for the semi-synthesis of paclitaxel is found in U.S. Pat. No. 5,750,737 to Sisti et al. In that patent, C7-CBZ baccatin III of the formula

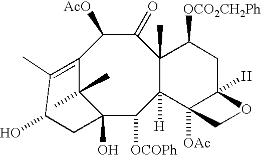

is esterified with a C3-N-CBZ-C2-O-protected (2R,3S)-3-phenylisoserine side chain of the formula:

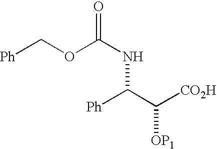

followed by deprotection, and 3N benzoylation to produce paclitaxel.

Another taxane compound that has been found to exhibit anti-tumor activity is the compound known as “docetaxel.” This compound is also sold under the trademark TAXOTERE®, the registration of which is owned by Sanofi Aventis. Docetaxel has the formula as follows:

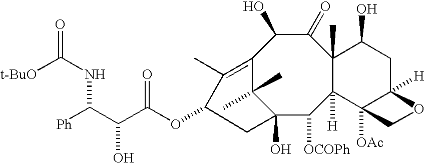

As may be seen in this formulation, docetaxel is similar to paclitaxel except for the inclusion of the t-butoxycarbonyl (Boc) group at the C3′ nitrogen position of the phenylisoserine side chain and a free hydroxyl group at the C10 position. Similar to paclitaxel, the synthesis of docetaxel is difficult due to the hindered C13 hydroxyl in the baccatin III backbone, which is located within the concave region of the hemispherical taxane skeleton. Several syntheses of docetaxel and related compounds have been reported in the Journal of Organic Chemistry: 1986, 51, 46; 1990, 55, 1957; 1991, 56, 1681; 1991, 56, 6939; 1992, 57, 4320; 1992, 57, 6387; and 993, 58, 255; also, U.S. Pat. No. 5,015,744 issued May 14, 1991 to Holton describes such a synthesis. Additional techniques for the synthesis of docetaxel are discussed, for example, in U.S. Pat. No. 5,688,977 to Sisti et al., U.S. Pat. No. 6,107,497 to Sisti et al.

Due to the promising anti-tumor activity exhibited by both paclitaxel and docetaxel, further investigations have indicated that analogs and derivates within the taxane family may lead to new and better drugs having improved properties such as increased biological activity, effectiveness against cancer cells that have developed multi-drug resistance (MDR), fewer or less serious side effects, improved solubility characteristics, better therapeutic profile and the like.

While the existing techniques for synthesizing paclitaxel and docetaxel certainly have merit, there is still a need for improved chemical processes that can produce this anti-cancer compound. Additionally, there is a need to provide new taxane compounds having improved biological activity for use in treating cancer and efficient protocols of forming these compounds. Particularly, there is a need for a new side chain that is easily and efficiently coupled to a taxane backbone for the synthesis of important pharmaceutical compounds and intermediates. The present invention is directed to meeting these needs.

### SUMMARY OF THE EXEMPLARY EMBODIMENTS

According to the present invention, then, methods are described for use in producing taxanes, taxane analogs, and derivatives thereof. Broadly, the method includes reacting a first compound of the general formula:

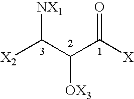

with a second compound of the general structure:

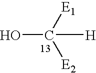

to give a third compound of the general formula:

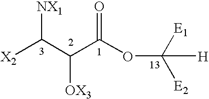

wherein:
X is a halogen or OR₄;
X₁ is either R₁R₂; R₁P₁; R₂P₁; or P₁P₁
X₂ is a substituted or unsubstituted: alkyl, alkenyl, aryl, aralkyl, or acyl;
X₃ is either R₁; R₂; or P₂;
R₁ and R₂ are independently H or substituted or unsubstituted: alkyl, alkenyl, aryl, aralkyl, or acyl;
R₄ is H, a substituted or unsubstituted: alkyl, alkenyl, aryl, aralkyl, acyl, alcoxy carbonyl or aryloxy carbonyl;
P₁ is an amine protecting group;
P₂ is a hydroxyl protecting group; and
E₁, E₂ and the carbon to which they are attached define a tetracyclic taxane nucleus.
   This third compound may take the more specific formula:

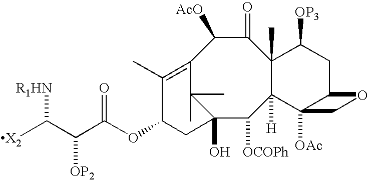

This third compound can be then converted to paclitaxel.

The second compound may have the a structure

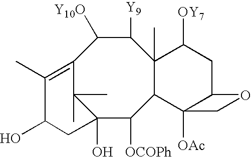

Y₇ is R₇; P₃; or Z₇;
Y₉ is H; hydroxyl; a ketone; OR₉; P₄; or Z₉;
Y₁₀ is R₁₀; P₅; or Z₁₀;
Z₇ is P₃ and together with Y₉ forms a cyclic structure when Y₉ is P₄;
Z₉ is either:
   P₄ and together with Y₇ forms a cyclic structure when Y₇ is P₃; or P₅ and together with Y₁₀ forms a cyclic structure when Y₁₀ is P₄;
Z₁₀ is P₅ and together with Y₉ forms a cyclic structure when Y₉ is P₄;
R₇ is H, substituted or unsubstituted: alkyl, alkenyl, aryl, aralkyl, or acyl;
R₉ is a substituted or unsubstituted: alkyl, alkenyl, aryl, aralkyl, or acyl;
R₁₀ is H, substituted or unsubstituted: alkyl, alkenyl, aryl, aralkyl, or acyl;
P₃ is a hydroxyl protecting group;
P₄ is a hydroxyl protecting group; and
P₅ is a hydroxyl protecting group.
   Here, if desired, X is a halogen; X₁ is R₁P₁; X₂ is Ph; X₃ is P₂; Y₇ is P₃; Y₉ is a ketone; Y₁₀ is P₅; R₁ is H; P₁ is Boc; P₂ is BOM; P₃ is Cbz; and P₅ is Cbz. Alternatively, X is fluorine; X₁ is R₁P₁; X₂ is Ph; X₃ is P₂; Y₇ is P₃; Y₉ is a ketone; Y₁₀ is P₅; R₁ is H; P₁ is Cbz; P₂ is BOM; P₃ is Cbz; and P₅ is Cbz. In another alternative, X is OR₄; X₁ is R₁P₁; X₂ is isobutyl; X₃ is P₂; Y₇ is P₃; Y₉ is a ketone; Y₁₀ is P₅; R₁ is H; R₄ is H; P₁ is Boc; P₂ is BOM; P₃ is Cbz; and P₅ is Cbz. In yet another alternative, X is a halogen; X₂ is isobutyl; Y₇ is P₃; Y₉ is a ketone; Y₁₀ is P₅; R₁ and R₂ are independently H or substituted or unsubstituted: alkyl, alkenyl, aryl, aralkyl, or acyl; R₃ is H; P₁ is Boc; P₂ is BOM; P₃ is Cbz; and P₅ is Cbz.

The third compound can have the formula:

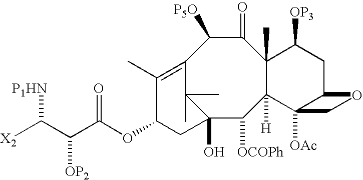

Here, the method can include the step of converting this third compound to docetaxel, paclitaxel, or a 7,9-acetal linked analog. This method may include the step of deprotecting the third compound by substituting hydrogen for P₁, P₂, P₃ and P₅ to form a fourth compound having the formula:

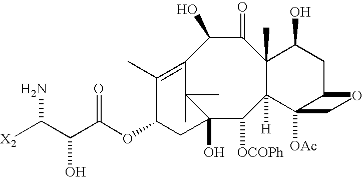

The third compound can also be deprotected by substituting hydrogen for P₃, and P₅ to form a fourth compound having the formula:

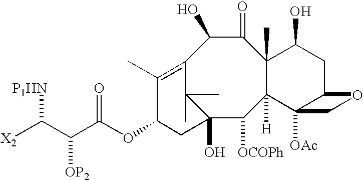

This fourth compound may be selectively acyalated at the C-10 position to form a fifth compound having the formula:

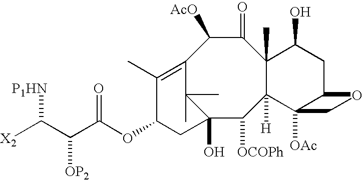

The method contemplates converting this fifth compound into paclitaxel.

The third compound can also be oxidized to form a fourth compound of the formula:

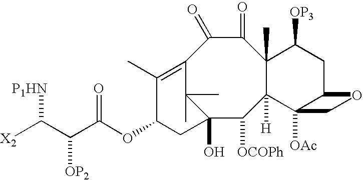

This fourth compound can then be reduced to form a fifth compound of the formula:

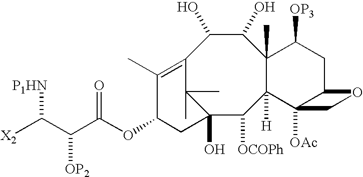

This fifth compound may be acylated at the C-10 position to form a sixth compound of the formula:

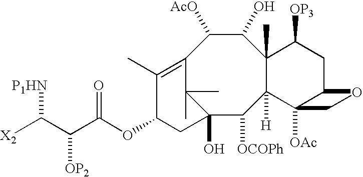

This sixth compound may further be deprotected by substituting hydrogen for P₃ thereby to form a seventh compound of the formula:

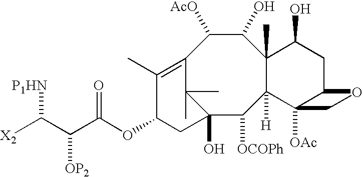

The seventh compound can be converted into an eighth compound of the formula:

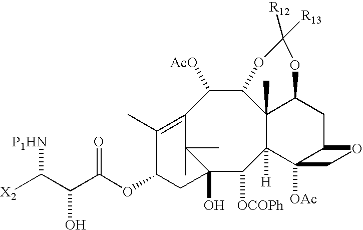

wherein R₁₂ and R₁₃ are independently H; substituted or unsubstituted: alkyl; alkenyl; aryl; aralkyl; or acyl. Here, R₁₂ and R₁₃ may each be independently selected from the group consisting of:

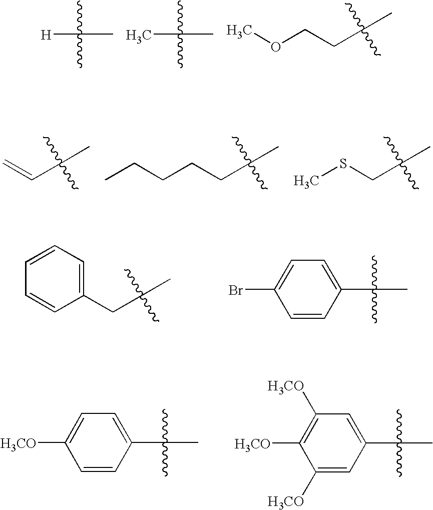

According to some embodiments of the invention, the first compound is a cyclic structure wherein the C-3 Nitrogen and the C-2 Oxygen are linked by a common protecting group that includes R₁ and R₂ and that has the formula:

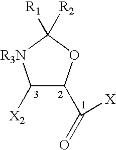

such that the third compound is a cyclic structure having the formula:

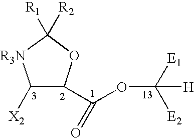

wherein R₃ is either H or P₁.
The second compound again may have the a structure

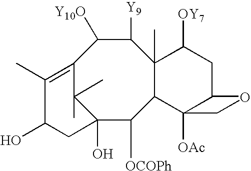

Y₇ is R₇; P₃; or Z₇;
Y₉ is H; hydroxyl; a ketone; OR₉; P₄; or Z₉;
Y₁₀ is R₁₀; P₅; or Z₁₀;
Z₇ is P₃ and together with Y₉ forms a cyclic structure when Y₉ is P₄;
Z₉ is either:
   P₄ and together with Y₇ forms a cyclic structure when Y₇ is P₃; or P₅ and together with Y₁₀ forms a cyclic structure when Y₁₀ is P₄;
Z₁₀ is P₅ and together with Y₉ forms a cyclic structure when Y₉ is P₄;
R₇ is H, substituted or unsubstituted: alkyl, alkenyl, aryl, aralkyl, or acyl;
R₉ is a substituted or unsubstituted: alkyl, alkenyl, aryl, aralkyl, or acyl;
R₁₀ is H, substituted or unsubstituted: alkyl, alkenyl, aryl, aralkyl, or acyl;
P₃ is a hydroxyl protecting group;
P₄ is a hydroxyl protecting group; and
P₅ is a hydroxyl protecting group.
   Here, if desired, X is a halogen; X₁ is R₁P₁; X₂ is Ph; X₃ is P₂; Y₇ is P₃; Y₉ is a ketone; Y₁₀ is P₅; R₁ is H; P₁ is Boc; P₂ is BOM; P₃ is Cbz; and P₅ is Cbz. Alternatively, X is fluorine; X₁ is R₁P₁; X₂ is Ph; X₃ is P₂; Y₇ is P₃; Y₉ is a ketone; Y₁₀ is P₅; R₁ is H; P₁ is Cbz; P₂ is BOM; P₃ is Cbz; and P₅ is Cbz. In another alternative, X is OR₄; X₁ is R₁P₁; X₂ is isobutyl; X₃ is P₂; Y₇ is P₃; Y₉ is a ketone; Y₁₀ is P₅; R₁ is H; R₄ is H; P₁ is Boc; P₂ is BOM; P₃ is Cbz; and P₅ is Cbz. In yet another alternative, X is a halogen; X₂ is isobutyl; Y₇ is P₃; Y₉ is a ketone; Y₁₀ is P₅; R₁ and R₂ are independently H or substituted or unsubstituted: alkyl, alkenyl, aryl, aralkyl, or acyl; R₃ is H; P₁ is Boc; P₂ is BOM; P₃ is Cbz; and P₅ is Cbz.

The present invention also discloses novel compounds produced in the foregoing methods. One such compound has the formula:

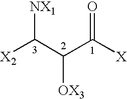

wherein:
X is a halogen or OR₄;
X₁ is either R₁, R₂; R₁P₁; R₂P₁; or P₁P₁
X₂ is a substituted or unsubstituted: alkyl, alkenyl, aryl, aralkyl, or acyl;
X₃ is either R₁; R₂; or P₂;
R₁ and R₂ are independently H or substituted or unsubstituted: alkyl, alkenyl, aryl, aralkyl, or acyl;
R₄ is a H, a substituted or unsubstituted: alkyl, alkenyl, aryl, aralkyl, acyl, alcoxy carbonyl or aryloxy carbonyl;
P₁ is an amine protecting group;
P₂ is a hydroxyl protecting group.
   More specifically, X can be selected from the group consisting of chlorine, bromine, fluorine, and iodine.

This compound may be a cyclic structure wherein the C-3 Nitrogen and the C-2 Oxygen are linked by a common protecting group that includes R₁ and R₂ and that has the formula:

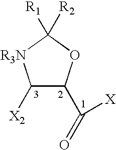

wherein R₃ is either H or P₁. X can again be selected from the group consisting of chlorine, bromine, fluorine, and iodine. If desired, X is chlorine; X₂ is isobutyl; R₁ and R₂ are independently H, or substituted or unsubstituted: alkyl, alkenyl, aryl, aralkyl or acyl; R₃ is P₁; and P₁ is Boc. Here, the compound may take the structural formula:

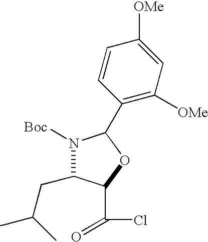

Alternatively, X is R₄; X₂ is isobutyl; R₁ and R₂ are independently H, or substituted or unsubstituted: alkyl, alkenyl, aryl, aralkyl or acyl; R₃ is P₁ R₄ is trimethylacetyl; and P₁ is Boc. Accordingly, another cyclic structure for this compound has the structural formula:

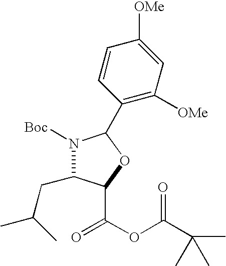

Where the compound has the general formula:

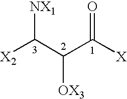

X can be OR₁₁; X₁ can be R₁P₁; X₂ can be isobutyl; X₃ can be P₂; R₁ is H; R₁₁ can be H; P₁ can be Boc; and P₂ can be BOM. Accordingly, the compound can have the structural formula:

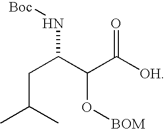

In the compound of the general formula, X can be fluorine; X₁ can be R₁P₁; X₂ can be isobutyl; X₃ can be P₂; R₁ is H; P₁ can be Boc; and P₂ can be BOM. Accordingly, the compound can have the structural formula:

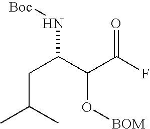

In the compound of the general formula, X can be OR₁₁; X₁ can be R₁P₁; X₂ can be Ph; X₃ can be P₂; R₁ can be H; R₁₁ can be H; P₁ can be Cbz; and P₂ can be BOM. Accordingly, the compound can have the structural formula:

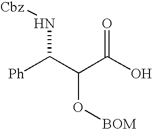

In the compound of the general formula, X can be fluorine; X₁ can be R₁P₁; X₂ can be Ph; X₃ can be P₂; R₁ can be H; P₁ can be Cbz; and P₂ can be BOM. Accordingly, the compound can have the structural formula:

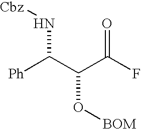

These and other aspects of the exemplary embodiments of the present invention will become more readily appreciated and understood from a consideration of the following detailed description when taken together with the accompanying drawings, in which:

### BRIEF DESCRIPTION OF THE FIGURES

FIG. 1 is a diagram of a generalized coupling reaction Schemes 1a, 1b and 1c according to the present invention;

FIG. 2 is a diagram of a generalized Scheme 2 for the synthesis of docetaxel from a coupled product formed by the coupling reaction generally shown in Scheme 1c;

FIG. 3 is a diagram of a generalized reaction Scheme 3 for the synthesis of paclitaxel from a coupled product formed by the coupling reaction generally shown in Scheme 1c;

FIG. 4 is a diagram of a generalized alternative reaction Scheme 4 for the synthesis of paclitaxel from a coupled product formed by the coupling reaction generally shown in Scheme 1c;

FIG. 5 is a diagram of a generalized reaction Scheme 5 for the synthesis of 9,10-α,α-7,9 acetal taxane analogs from a coupled product formed by the coupling reaction generally shown in Scheme 1c;

FIG. 6 is a diagram of an exemplary synthesis of docetaxel according to the present invention;

FIG. 6a is a diagram of another exemplary synthesis of docetaxel according to the present invention;

FIG. 7 is a diagram of an exemplary synthesis of paclitaxel according to the present invention;

FIG. 8 is a diagram of another exemplary synthesis of paclitaxel according to the present invention;

FIG. 9 is a diagram of an exemplary synthesis of 9,10-α,α-7,9 acetal taxane analogs according to the present invention;

FIG. 10 is a diagram of a coupling reaction according to the general scheme shown in FIG. 1b;

FIG. 11 is a diagram of an alternative coupling reaction according to the general scheme shown in FIG. 1b;

FIG. 12 is a diagram of an exemplary synthesis of two side chain compounds according to the present invention;

FIG. 13 is a diagram of an exemplary synthesis of an alternative side chain compound according to the present invention; and

FIG. 14 is a diagram of an exemplary synthesis of yet another side chain compound according to the present invention.

### DETAILED DESCRIPTION OF THE EXEMPLARY EMBODIMENTS

As used above, and throughout the description of the invention, the following terms, unless otherwise indicated, shall be understood to have the following meanings:

Alkyl

The term “alkyl” as used herein alone or as part of another group, denotes optionally substituted, straight and branched chain saturated hydrocarbon groups, preferably having 1 to 12 carbons in the normal chain.

The term “substituted alkyl” refers to an alkyl group substituted by, for example, one to four substituents, such as, halo, trifluoromethyl, trifluoromethoxy, hydroxy, alkoxy, cycloalkyoxy, heterocylooxy, oxo, alkanoyl, aryloxy, alkanoyloxy, amino, alkylamino, arylamino, aralkylamino, cycloalkylamino, heterocycloamino, disubstituted amines in which the 2 amino substituents are selected from alkyl, aryl or aralkyl, alkanoylamino, aroylamino, aralkanoylamino, substituted alkanoylamino, substituted arylamino, substituted aralkanoylamino, thiol, alkylthio, arylthio, aralkylthio, cycloalkylthio, heterocyclothio, alkylthiono, arylthiono, aralkylthiono, alkylsulfonyl, arylsulfonyl, aralkylsulfonyl, sulfonamido (e.g. SO.sub.2 NH.sub.2), substituted sulfonamido, nitro, cyano, carboxy, carbamyl (e.g. CONH.sub.2), substituted carbamyl (e.g. CONH alkyl, CONH aryl, CONH aralkyl or cases where there are two substituents on the nitrogen selected from alkyl, aryl or aralkyl), alkoxycarbonyl, aryl, substituted aryl, guanidino and heterocyclos, such as, indolyl, imidazolyl, furyl, thienyl, thiazolyl, pyrrolidyl, pyridyl, pyrimidyl and the like. Where noted above where the substituent is further substituted it will be with halogen, alkyl, alkoxy, aryl or aralkyl.

Exemplary unsubstituted such groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl and the like. Exemplary substituents may include one or more of the following groups: halo, alkoxy, alkylthio, alkenyl, alkynyl, aryl, cycloalkyl, cycloalkenyl, hydroxy or protected hydroxy, carboxyl (—COOH), alkyloxycarbonyl, alkylcarbonyloxy, carbamoyl (NH.sub.2-CO—), amino (—NH.sub.2), mono- or dialkylamino, or thiol (—SH).

Alkenyl

The term “alkenyl”, as used herein alone or as part of another group, denotes such optionally substituted groups as described for alkyl, further containing at least one carbon to carbon double bond. Exemplary substituents include one or more alkyl groups as described above, and/or one or more groups described above as alkyl substituents.

Aryl

The term “aryl”, as used herein alone or as part of another group, denotes optionally substituted, homocyclic aromatic groups, preferably containing 1 or 2 rings and 6 to 12 ring carbons. Exemplary unsubstituted such groups include phenyl, biphenyl, and naphthyl. Exemplary substituents include one or more, preferably three or fewer, nitro groups, alkyl groups as described above, and/or groups described above as alkyl substituents.

The term “substituted aryl” refers to an aryl group substituted by, for example, one to four substituents such as alkyl; substituted alkyl, halo, trifluoromethoxy, trifluoromethyl, hydroxy, alkoxy, cycloalkyloxy, heterocyclooxy, alkanoyl, alkanoyloxy, amino, alkylamino, aralkylamino, cycloalkylamino, heterocycloamino, dialkylamino, alkanoylamino, thiol, alkylthio, cycloalkylthio, heterocyclothio, ureido, nitro, cyano, carboxy, carboxyalkyl, carbamyl, alkoxycarbonyl, alkylthiono, arylthiono, alkysulfonyl, sulfonamido, aryloxy and the like. The substituent may be further substituted by halo, hydroxy, alkyl, alkoxy, aryl, substituted aryl, substituted alkyl or aralkyl.

Aralkyl

The term “aralkyl”, as used herein alone or as part of another group refers to alkyl groups as discussed above having an aryl substituent, such as benzyl or phenethyl, or naphthylpropyl, or an aryl as defined above.

Acyl

The term “acyl”, as used herein alone or as part of another group, denotes the moiety formed by removal of the hydroxyl group from the group —COOH of an organic carboxylic acid. The acyl group can specifically be PhCO or BnCO, for example.

Hydroxyl Protecting Group

The term “hydroxy (or hydroxyl) protecting group”, as used herein, denotes any group capable of protecting a free hydroxyl group which, subsequent to the reactions for which it is employed, may be removed without destroying the remainder of the molecule. Such groups, and the synthesis thereof, may be found in “Protective Groups in Organic Synthesis” by T. W. Greene and P. G. M. Wuts, Protective Groups in Organic Synthesis, 3rd Edition, John Wiley & Sons, New York (1999), or Fieser & Fieser. Exemplary hydroxyl protecting groups include methoxymethyl, 1-ethoxyethyl, 1-methoxy-1-methylethyl, benzyloxymethyl, (.beta.-trimethylsilyl-ethoxy)methyl, tetrahydropyranyl, benzyloxycarbonyl, 2,2,2-tri-chloroethoxycarbonyl, t-butyl(diphenyl)silyl, trialkylsilyl, trichloromethoxycarbonyl, and 2,2,2-trichloroethoxymethyl.

Amine Protecting Group

The term “amine protecting group” as used herein means an easily removable group which is known in the art to protect an amino group against undesirable reaction during synthetic procedures and to be selectively removable. The use of amine protecting groups is well known in the art for protecting groups against undesirable reactions during a synthetic procedure and many such protecting groups are known, for example, T. W. Greene and P. G. M. Wuts, Protective Groups in Organic Synthesis, 3rd Edition, John Wiley & Sons, New York (1999), incorporated herein by reference. Exemplary amine protecting groups are acyl, including formyl, acetyl, chloroacetyl, trichloroacetyl, o-nitrophenylacetyl, o-nitrophenoxyacetyl, trifluoroacetyl, acetoacetyl, 4-chlorobutyryl, isobutyryl, o-nitrocinnamoyl, picolinoyl, acylisothiocyanate, aminocaproyl, benzoyl and the like, and acyloxy including methoxycarbonyl, 9-fluorenylmethoxycarbonyl, 2,2,2-trifluoroethoxycarbonyl, 2-trimethylsilylethxoycarbonyl, vinyloxycarbonyl, allyloxycarbonyl, t-butyloxycarbonyl (BOC), 1,1-dimethylpropynyloxycarbonyl, benzyloxycarbonyl (CBZ), p-nitrobenzyloxycarbony, 2,4-dichlorobenzyloxycarbonyl, and the like.

Halogen

The term “halogen” as used herein alone or as part of another group, denotes chlorine, bromine, fluorine, and iodine.

Taxane

The term “taxane”, as used herein, denotes compounds containing a taxane moiety as described above. The term “C-13 acyloxy sidechain-bearing taxane”, as used herein, denotes compounds containing a taxane moiety as described above, further containing an acyloxy sidechain directly bonded to said moiety at C-13 through the oxygen of the oxy group of the acyloxy substituent.

The exemplary embodiments of the present invention generally relate to the synthesis of anti-tumor compounds including, for example, docetaxel, paclitaxel, and taxane analogs having a stereochemistry at the C-9 and C-10 OH positions. One aspect of the present invention is a novel and useful side chain for attachment to a taxane backbone for the synthesis of these anti-tumor compounds. Another aspect of the present invention includes the synthesis of desired anti-tumor compounds subsequent to the attachment of the novel side chain to the taxane backbone.

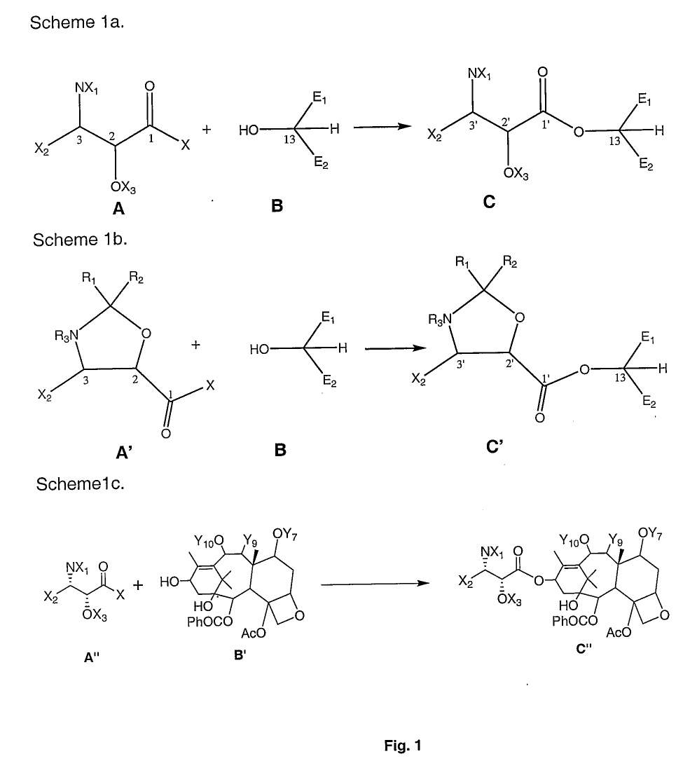

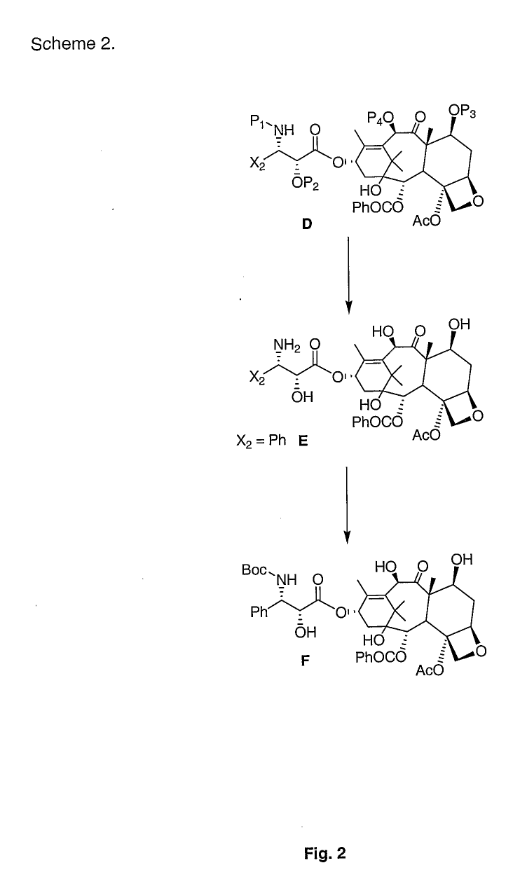

Turning first, then, to FIG. 1, (Schemes 1a, b and c), this new side chain is generally represented as compound A. As shown, side chain A may be attached at the C13 position of taxane backbone B, thereby to form coupled product C. Coupled product C may then, if desired, undergo further synthesis to produce the anti-tumor compounds of interest, such as generally shown in FIGS. 2-5 (Schemes 2-5), which will be discussed in more detail below.

Broadly, side chain A may have the formula wherein:

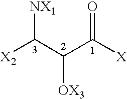

X is a halogen or OR₄;
X₁ is either R₁R₂; R₁P₁; R₂P₁; or P₁P₁
X₂ is substituted or unsubstituted: alkyl, alkenyl, aryl, aralkyl, or acyl;
X₃ is either R₁; R₂; or P₂;
R₁ and R₂ are independently H or substituted or unsubstituted: alkyl, alkenyl, aryl, aralkyl, or acyl;
R₄ is a H, a substituted or unsubstituted: alkyl, alkenyl, aryl, aralkyl, acyl, alkoxy carbonyl or aryloxy carbonyl
P₁ is an amine protecting group;
P₂ is a hydroxyl protecting group;

Side chain A can also have a structure as follows, when 2-O and 3-N are linked with a common protecting group such as in a cyclic acetal:

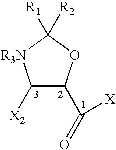

wherein, R₃ is either H or P₁

Some examples of side chain A have the following exemplary structural formulas:

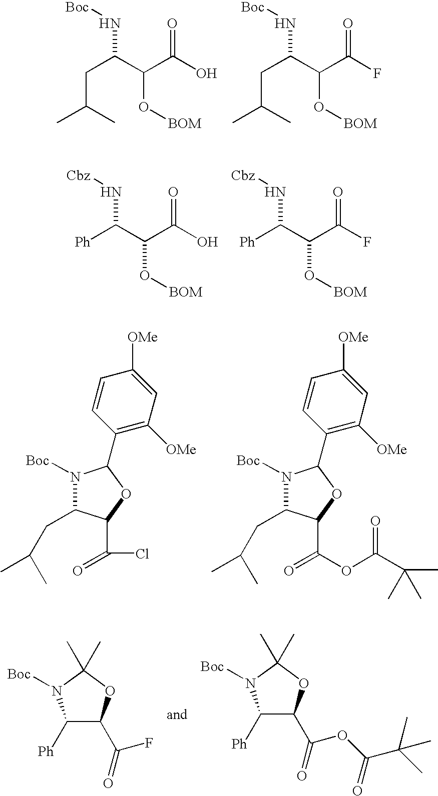

Broadly taxane backbone B may have the formula wherein:

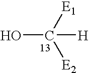

E1, E2 and the carbon to which they are attached define a tetracyclic taxane nucleus
Taxane backbone B may have the following general structural formula wherein:

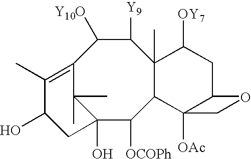

Y₇ is R₇; P₃; or Z₇;
Y₉ is H; hydroxyl; a ketone; OR₉; P₄; or Z₉;
Y₁₀ is R₁₀; P₅; or Z₁₀;
Z₇ is P₃ and together with Y₉ forms a cyclic structure when Y₉ is P₄;
Z₉ is either:
   P₄ and together with Y₇ forms a cyclic structure when Y₇ is P₃; or P₅ and together with Y₁₀ forms a cyclic structure when Y₁₀ is P₄;
Z₁₀ is P₅ and together with Y₉ forms a cyclic structure when Y₉ is P₄;
R₇ is H, substituted or unsubstituted: alkyl, alkenyl, aryl, aralkyl, or acyl;
R₉ is a substituted or unsubstituted: alkyl, alkenyl, aryl, aralkyl, or acyl;
R₁₀ is H, substituted or unsubstituted: alkyl, alkenyl, aryl, aralkyl, or acyl;
P₃ is a hydroxyl protecting group;
P₄ is a hydroxyl protecting group; and
P₅ is a hydroxyl protecting group.
   When side chain A is coupled with taxane backbone B, coupled product C has the following broad structure;

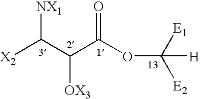

Wherein: X₁, X₂, X₃, E₁ and E₂ are as above

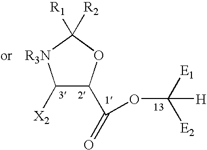

When 2-O and 3-N are linked with a common protecting group; wherein R₁ and R₂ are as above.
Some examples of coupled products have the following exemplary structural formulas:

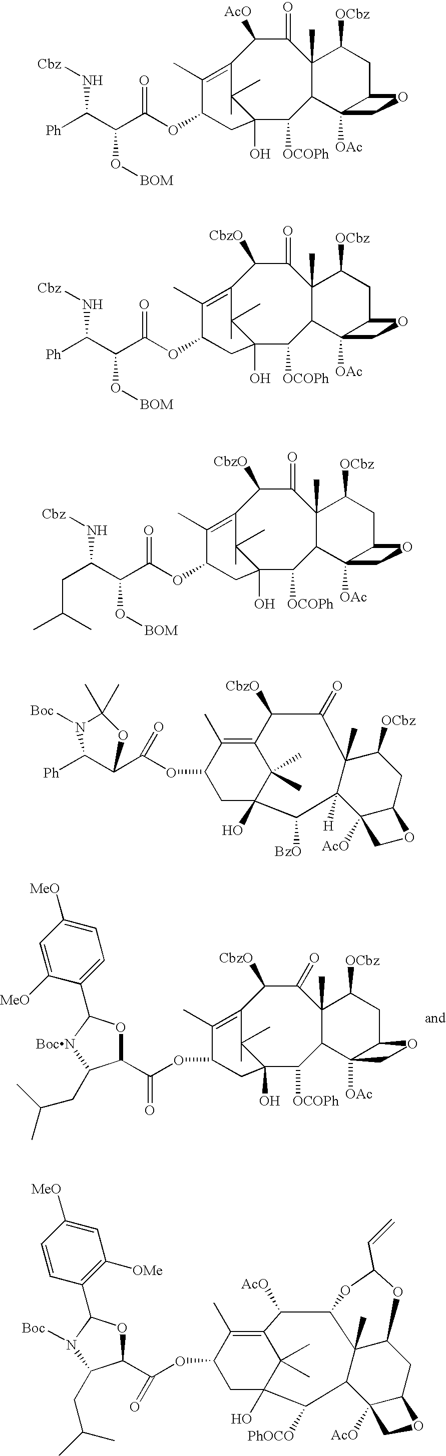

Set forth below are general examples, followed by specific examples, of both the synthesis of coupled product C as well as the subsequent anti-tumor compounds and intermediates formed thereby. It should be appreciated however, that coupled product C could be useful to synthesize other useful compounds.

### I. Synthesis of Docetaxel

Docetaxel may be formed in a number of ways according to the present invention, a general example of which is shown in FIG. 2 (Scheme 2). As shown, coupled product D, which is formed by the attachment of a side chain to a taxane backbone as generally shown in Scheme 1c, undergoes various transformations to form docetaxel F. More particularly, coupled product D is first deprotected at the C7, C10, C3′N and C2′ to form a first intermediate E. Subsequently, the Boc group is attached to the N—C3′ site to form docetaxel F.

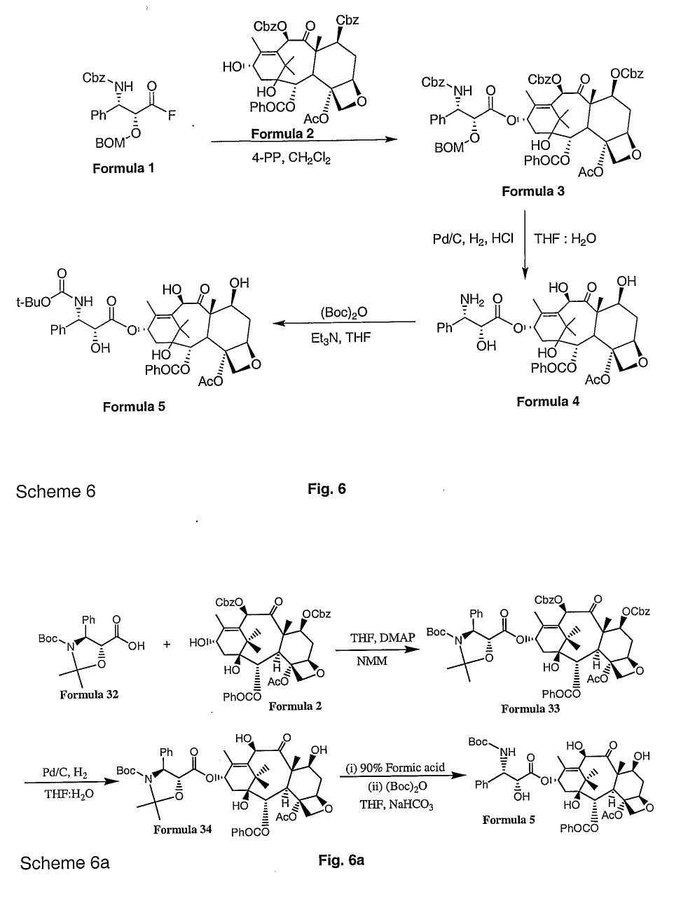

Such a process is exemplified in FIG. 6. As shown, side chain of Formula 1 (wherein: X is fluorine; X₁ is R₁P₁; X₂ is Ph; X₃ is P₂; R₁ is H; P₁ is Cbz; and P₂ is BOM) is coupled to taxane backbone of Formula 2, which is C7, C10 di-Cbz 10-deacetylbaccatin III (wherein: Y₇ is P₃; Y₉ is a ketone; Y₁₀ is P₅; P₃ and P₅ are each Cbz) to form coupled product of Formula 3.

A solution of the acid fluoride, Formula 1, in methylene chloride was added via a syringe, to a solution of C7, C10 di-Cbz 10-deacetylbaccatin III, Formula 2, (5.6 g) and 4-PP (1.55 g) in anhydrous methylene chloride (40 mL), at room temperature and an atmosphere of nitrogen. The reaction was stirred at room temperature for four hours, then, diluted with methylene chloride (75 mL), washed with water (2×50 mL), brine (1×30 mL), dried over sodium sulphate and rotostripped. The crude product was purified on a silica plug, eluting with a gradient eluent involving isopropyl acetate and heptanes. The pure fractions were pooled and rotostripped to give the cleaned-up coupled ester as a foamy solid. The solid was suspended in methanol (200 mL) and stirred vigorously for five hours at room temperature. The white solids were filtered, washed with minimum methanol and dried in the vacuum oven to afford the coupled ester, Formula 3, as a white solid (7.3 g, 86%).

A solution of HCl (1.7 mL) in tetrahydrofuran (25 mL) and water (1.7 mL) and Pd/C (10 wt % palladium, 4.0 g) was added to a solution of coupled ester, Formula 3, (5.0 g) in tetrahydrofuran (75 mL). The reaction was stirred vigorously overnight under an atmosphere of hydrogen. The reaction mixture was then filtered through a bed of celite (15 g), washed with tetrahydrofuran (2×75 mL) and the filtrate was transferred to a round bottomed flask and used as such for the next reaction.

To this tetrahydrofuran solution was added di-tert-butyldicarbonate (2.0 g) and triethylamine (3.5 mL) at room temperature under nitrogen atmosphere and stirred overnight. The reaction mixture was then filtered through a bed of celite and washed with isopropyl acetate (3×75 mL). The organic layer was then washed with 0.1N HCl solution (till neutral pH), water (2×50 mL), dried and rotostripped to afford docetaxel (4.26 g), Formula 5, which is then purified by column chromatography.

### II. Synthesis of Paclitaxel

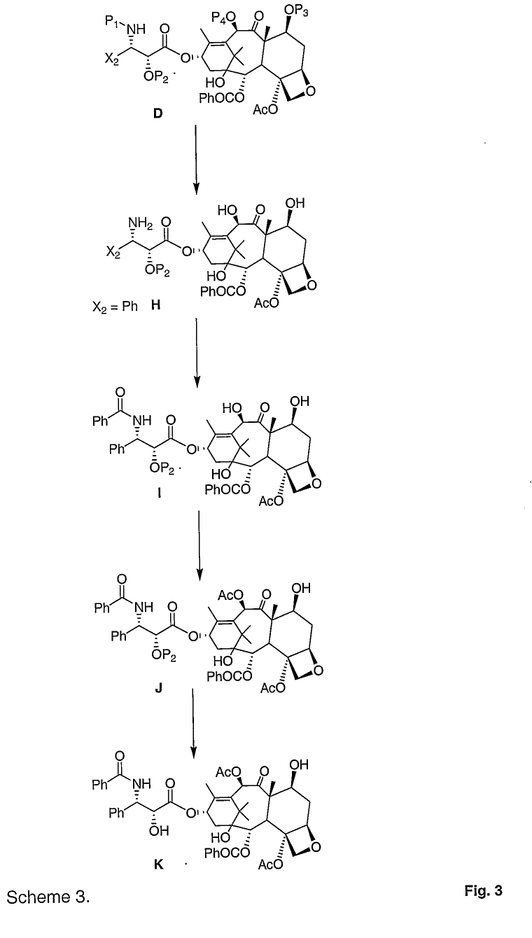

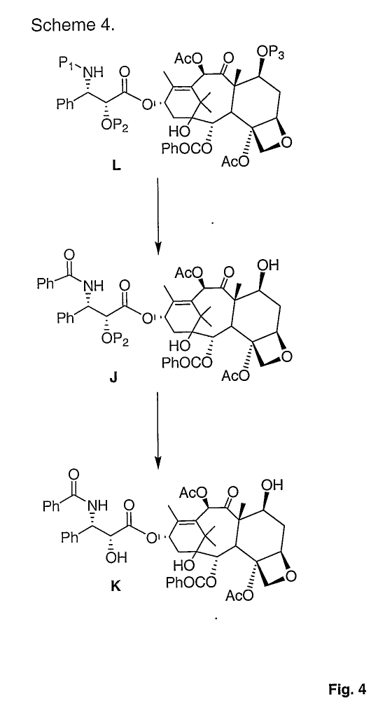

Two general syntheses of paclitaxel are shown, the first in FIG. 3 (Scheme 3) and an alternative in FIG. 4 (Scheme 4). Turning first to FIG. 3, coupled product D is, as described above, generally formed by the attachment of a side chain to a taxane backbone as generally shown in Scheme 1c. The protecting groups are then removed at C7 and C10 and the C3′ nitrogen side chain site to produce intermediate compound H. Thereafter, intermediate compound H is acylated at the C3′ nitrogen, yielding intermediate compound I, and then selectively acylated at C10 site to yield intermediate compound J. Compound J is then deprotected at the C2′ site to produce paclitaxel K.

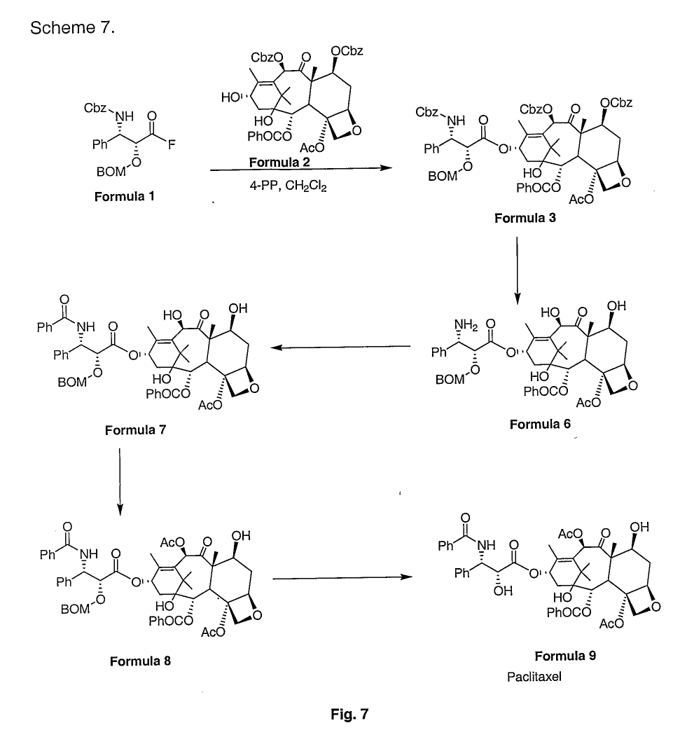

The general process shown in Scheme 3 may be further exemplified in FIG. 7. Here again, coupled ester of Formula 3 is formed by the coupling of side chain of Formula 1 to C7, C10 di-Cbz 10-deacetylbaccatin III of Formula 2 as described above with reference to FIG. 6. The transformation of coupled ester of Formula 3, through intermediate compounds of Formulas 6, 7, and 8, to arrive at paclitaxel of Formula 9 is described in U.S. Pat. No. 6,066,749 and U.S. Pat. No. 6,448,417, which are both herein incorporated by reference.

An alternative generalized scheme for producing paclitaxel is shown in FIG. 4 (Scheme 4), beginning with coupled product L, which can be formed by the generalized reaction shown in Scheme 1c. Coupled product L is first deprotected at C7 and the N—C3′ site and the benzoyl group is placed onto the nitrogen to yield intermediate compound J. The benzoyl group is then placed onto the nitrogen and deprotection at C2′ yields paclitaxel K.

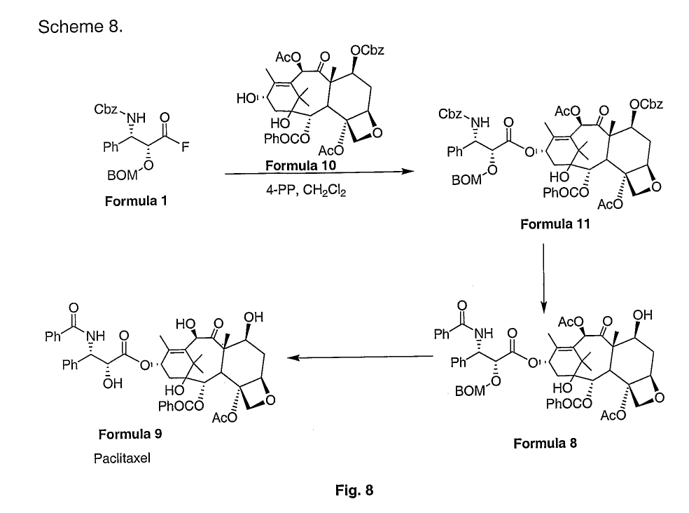

The process in Scheme 4 is exemplified in FIG. 8. As shown, side chain of Formula 1 (wherein: X is fluorine; X₁ is R₁P₁; X₂ is Ph; X₃ is P₂; R₁ is H; P₁ is Cbz; and P₂ is BOM) is coupled to taxane backbone of Formula 10, which is C7-Cbz baccatin III, Formula 10 (wherein: Y₇ is R₇; Y₉ is a ketone; Y₁₀ is P₅; R₁₀ is AcO; P₃ is Cbz) to form coupled product of Formula 11.

A solution of the acid fluoride, Formula 1, in methylene chloride was added through a syringe to a solution of C7-Cbz baccatin III, Formula 10, (3.93 g) and 4-PP (1.62 g) in anhydrous methylene chloride (40 mL) at room temperature the reaction was stirred under nitrogen atmosphere for four hours, diluted with methylene chloride (75 mL), washed with saturated ammonium chloride solution (1×50 mL), water (2×50 mL), brine (1×30 mL), dried over sodium sulphate and rotostripped to afford a foamy solid (˜8.9 g). The solid was suspended in methanol (30 mL) and stirred vigorously for five hours at room temperature. The white solids were filtered, washed with minimum methanol and dried in the vacuum oven to afford the coupled ester, Formula 11, as a white solid (4.9 g, 79% yield, 94.5% by area). The transformation of coupled ester of Formula 11 to the resultant paclitaxel of Formula 9 is described in U.S. Pat. No. 5,750,737 which is herein incorporated by reference.

### III. Synthesis of 7,9-Acetal Linked Analogs

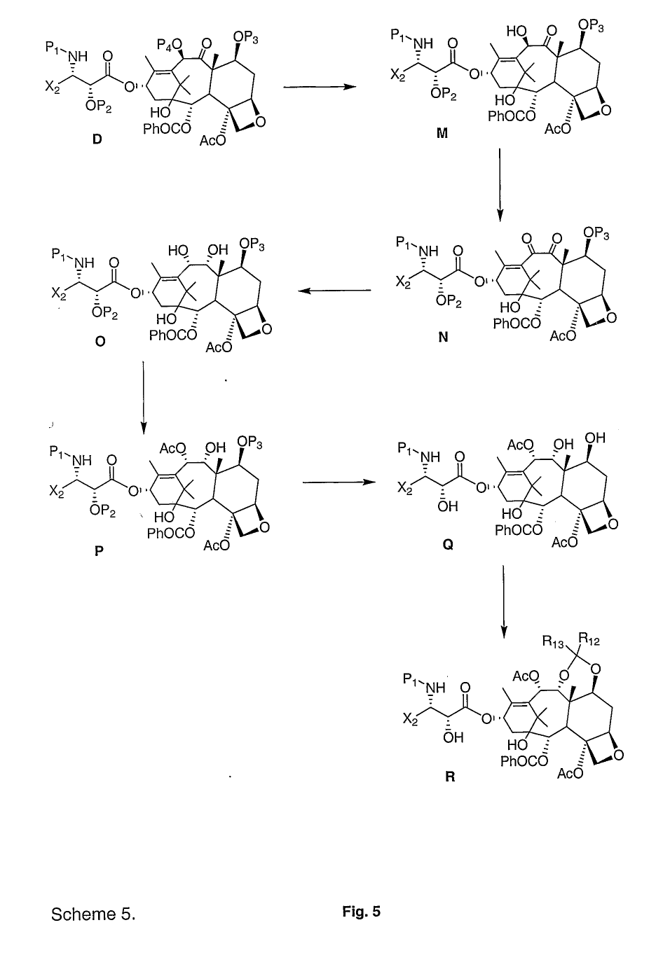

A general synthesis of 7,9-acetal linked analogs is shown in FIG. 5 (Scheme 5). Coupled product D, which is generally formed by a process according to Scheme 1c, and is synthesized to yield 7,9-acetal linked analog R. In general, coupled product D is deprotected at C10 to form intermediate product M, which is then oxidized to form intermediate compound N. Reduction of intermediate compound N yields intermediate compound O, which after selective acylation at C10 yields intermediate compound P. Intermediate compound P is then deprotected at both the C7 and the C2′ sites to afford intermediate compound Q, which was thereafter converted to 7,9-acetal linked analog R.

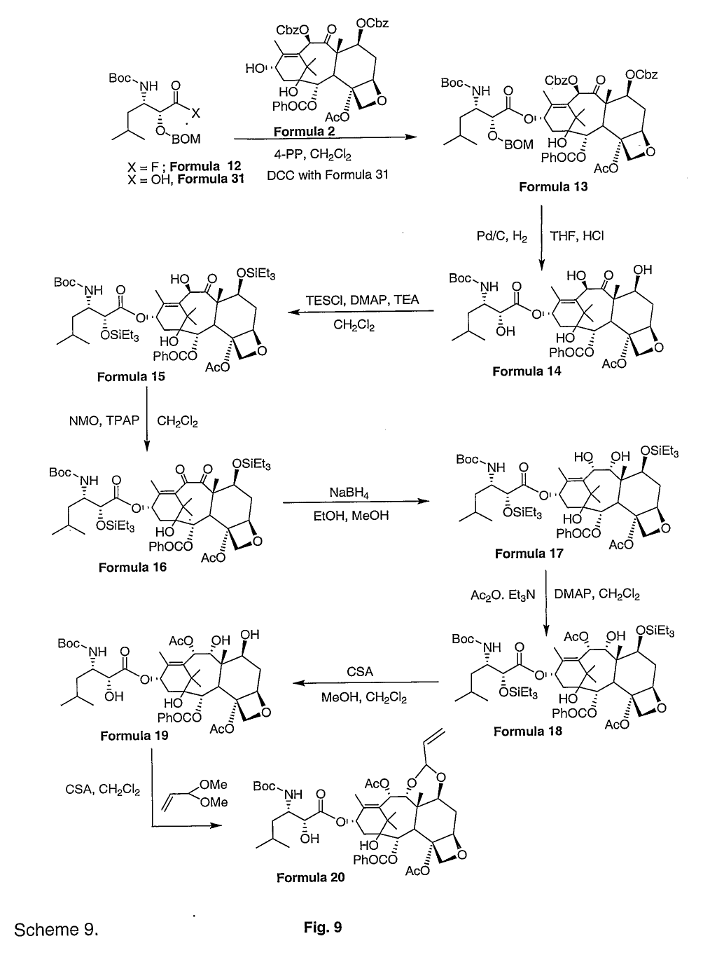

Such a process is exemplified in FIG. 9. As shown, side chain of Formula 31 (wherein: X is OR₄; X₁ is R₁P₁; X₂ is isobutyl; X₃ is P₂; R₁ is H; R₄ is H; PI is Boc; and P₂ is BOM) is coupled to C7, C10 di-Cbz 10-deacetylbaccatin III, Formula 2 to yield coupled ester of Formula 13. Here, the side chain of Formula 31, (38 g, 99.6 mmol) was dissolved in toluene to a known concentration (0.09524 g/mL). This solution was added to Formula 2 (54.0 g, 66.4 mmol). The solution was heated in a warm-water bath and DMAP (8.13 g, 66.4 mmol) and DCC (25.28 g, 119.6 mmol) in toluene (540 mL) were added to the warm reaction mixture. While maintaining the temperature at about 51° C., the reaction was continually stirred and sampled periodically for HPLC. After 3 hours, additional DCC (13.0 g) in toluene (140 mL) was added.

After approximately 25 hours, MTBE (450 mL) was added and the reaction mixture was filtered through a pad of silica gel, washed with MTBE followed by EtOAc, and concentrated to give 61.8 g oil. The silica was washed again with EtOAc and the second pool was concentrated to 50 mL and allowed to sit. The following day the second pool had started to crystallize. It was filtered and the solids were washed with 1:1 heptane/IPAc and dried under vacuum at 40° C. to give a solid of Formula 13.

Next, Formula 13 was deprotected at both the C7 and C10 positions and the C2′ side chain position to give Formula 14. A Parr reactor was charged with a solution of Formula 13 (68.0 g, 57.823 mmol) in THF (1.02 L). The reactor was flushed with nitrogen and a solution of HCl (24.75 mL) in THF (340 mL) was added followed by Pd/C (10%, wet type containing 50% water) (108.8 g). The reactor was evacuated and flushed with nitrogen repeatedly (thrice), followed by hydrogen (twice). The contents of the reactor were then stirred vigorously, overnight, at RT under hydrogen pressure (40 psi). The reaction was judged complete HPLC analysis. The contents of the reactor were then filtered through a pad of celite (celite 521, 100 g) and washed with THF. The green filtrate was neutralized with TEA (20 mL) to pH 7.5 and evaporated in-vacuo. The residue was dissolved in isopropyl acetate and washed with water. The emulsion formed, if any, was filtered through filter paper under suction and the filtrate was washed with saturated ammonium chloride solution and brine. The filtrate was then dried over anhydrous sodium sulfate and passed through a silica pad, eluting with isopropyl acetate. The solvents were rotostripped and the residue triturated with heptanes (twice) and rotostripped to afford the crude product which was purified on a silica column to afford clean Formula 14 as a white solid (40.64 g).

Formula 14 was then converted to Formula 15. Formula 14 (41.37 g, 52.5 mmol) was dissolved in DCM (500 mL) at room temperature. TEA (35 mL) followed by DMAP (1.284 g) and TES-CI (˜30 mL, 3.5 eq) were added to the solution and stirred. Additional TES-CI (15 mL) and TEA (20 mL) were added, and after 6 hours HPLC analysis indicated completion of the reaction.

The reaction was then quenched by the addition of EtOH (25 mL). The solvent was stripped to half the volume on the rotavapor and the residue was purified on a silica gel flash column eluting with 8:2 heptane/IPAc. Fractions containing the product were pooled and concentrated to give Formula 15 as a foam.

Formula 15 was then oxidized to form Formula 16. A solution of Formula 15 (24.45 g, 24.0 mmol) and 4-methyl morpholine N-oxide (10.1 g, 84 mmol) in DCM (340 mL) was dried over Na₂SO₄ for 1 hour and then filtered through 24 cm fluted filter paper into a 2 L 3-N round bottom flask. The Na₂SO₄ solids were washed with DCM (100 mL) into the flask. Molecular sieves (6.1 g, 15% wt/wt) were added to the stirring solution. TPAP (1.38 g) was added and the reaction was allowed to stir under a N₂ atmosphere. Samples were taken periodically for HPLC. Additional TPAP (0.62 g) was added after 2 hours and again (0.8 g) after 15 hours. The reaction mixture was applied to a pad of silica gel (86 g), wet with 8:2 heptane/IPAc and eluted with IPAc. The fractions were collected, pooled and concentrated to a foamy solid product of Formula 16 which was then recrystallized from methanol.

Formula 16 was then reduced to form Formula 17. NaBH₄ (365 mg, 6 eq) was added to a stirred solution of Formula 16 (1.6 g) in EtOH (19 mL) and MeOH (6.5 mL) at 0° c. After 1 hour, the reaction mixture was removed from the ice-water bath and at 2 hours, the reaction was sampled for HPLC, which indicated the reaction had gone to completion. The reaction mixture was cooled in an ice-water bath and quenched with a solution of NH₄OAc in MeOH (15 mL) followed by the addition of IPAc (50 mL) and H₂O (20 mL). The organic layer was separated and washed with water (20 mL) and brine (10 mL). It was dried over Na₂SO₄ and concentrated on the rotovap. It was placed in the vacuum oven to give product of Formula 17 as a foam.

Formula 17 was next acylated to form Formula 18. TEA (5.8 mL, 41.5 mmol), Ac₂O (2.62 mL, 27.7 mmol) and DMAP (724 mg, 5.5 mmol) were added to a solution of Formula 17 (14.1 g. 13.84 mmol) in DCM (50 mL). The reaction was stirred and sampled for HPLC periodically. At 19 hours, HPLC indicated the reaction had gone to completion. The reaction mixture was diluted with IPAc (300 mL) and poured into 5% NaHCO₃ (100 ml). The organic layer was then separated and washed with water (100 mL), saturated NH₄Cl (2×100 mL), water (3×50 mL) and brine (50 mL). The solution was dried over Na₂SO₄ and concentrated to give a foam product of Formula 18.

Next, Formula 18 was converted to a compound of Formula 19. To a solution of Formula 18 (3.0 g, 2.829 mmol) in DCM (24 mL) and MeOH (6 mL), at room temperature, CSA (0.0394 g, 0.17 mmol) was added. The reaction was judged complete at four hours by LCMS analysis. 5% NaHCO₃ (15 mL) was added to the reaction mixture and shaken vigorously in a separatory funnel and the layers were separated. The organic layer was washed with brine, dried over Na₂SO₄, and concentrated. MTBE (3×25 mL) was added and the reaction mixture was concentrated to dryness after each addition to finally give 3.7068 g foam. The foam was dissolved in MTBE (10 mL) and stirred. Heptane (50 mL) was slowly added to the reaction solution and solids began to form immediately. The solids were vacuum filtered and rinsed with heptane (70 mL). The solids were collected and dried in a vacuum oven at 40° C. to give Formula 19.

Formula 19 was then converted to Formula 20. A solution of Formula 19 (2.1 g, 2.52 mmol) in DCM (10.5 mL) was stirred at room temperature. Next, 3,3-dimethoxy-1-propene (2.03 g, 17.7 mmol) followed by CSA (0.035 g, 0.15 mmol) were added to the solution. After the solution was stirred for 3.5 hours, LCMS indicated the reaction had gone to completion. The reaction was diluted with DCM (25 mL) and transferred to a separatory funnel and washed with 55 mL 5% NaHCO₃ solution. The layers were separated and the aqueous layer was washed with DCM (25 mL). The two organic layers were combined, washed with brine, dried over Na₂SO₄ and concentrated. The crude product was purified by silica gel flash chromatography eluting with 50:50 MTBE/heptane. The fractions were collected, pooled, concentrated and dried in a vacuum oven at 50° C. to give product of Formula 20.

### IV. Alternative Side Chain Coupling Reactions

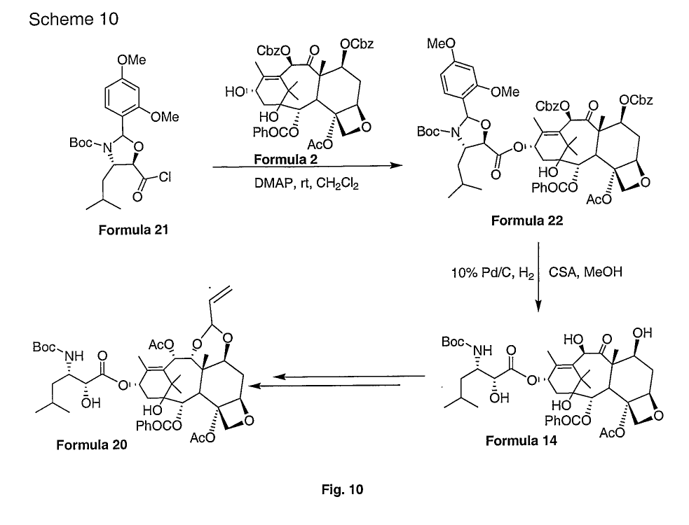

Additional specific examples of the coupling reaction generally shown in FIG. 1 (Scheme 1a, b and c) are shown in FIGS. 9, 10 and 11. With respect to FIG. 10, side chain of Formula 21 (wherein: X is chlorine; 2-O and 3-N are linked with a common protecting group; R₃ is P₁; R₁ and R₂ are H and substituted aryl; X₂ is isobutyl; P₁ is Boc) is coupled to C7, C10 di-Cbz 10-deacetylbaccatin III (wherein: Y₇ is P₃; Y₉ is a ketone; Y₁₀ is P₅; P₃ and P₅ are each Cbz) Formula 2 to form coupled product of Formula 22.

40 g of anhydrous sodium sulfate was added to a solution of C7, C10 di-Cbz 10-deacetylbaccatin III 5.00 g (6.15 mmol, 1.0 eq), Formula 2, in 150 mL dichloromethane. After three hours, the mixture was filtered and the filtrate was concentrated under reduced pressure. The C7, C10 di-Cbz 10-deacetylbaccatin III, Formula 2, was re-dissolved in anhydrous dichloromethane (50 mL) at ambient temperature, and subsequently, 2.25 g (18.4 mmol, 3.0 eq) 99% 4-DMAP was added and the solution was placed under an inert atmosphere of nitrogen. A solution of side chain, Formula 21, in dichloromethane, was added to the resulting solution at ambient temperature. The progress of the reaction was monitored by HPLC (a reaction aliquot was quenched into methanol). After stirring overnight, the solution was concentrated to dryness and the crude product was flash chromatographed over silica gel using 2/1 (v/v) EtOAc-heptane as the eluent. Appropriate fractions were pooled and concentrated in vacuo to constant weight to afford 7.31 g (98.7%) coupled product, Formula 22 as an off-white solid; 84.5 AP (230 nm).

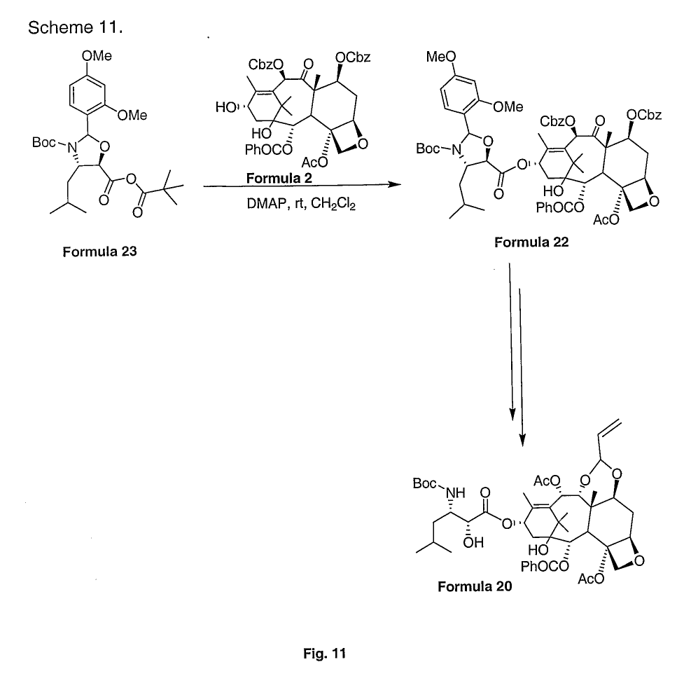

Turning now to FIG. 11, side chain of Formula 23 (wherein: X is OR₄; 2-O and 3-N are linked with a common protecting group; R₃ is P1; R₁ and R₂ are H and substituted aryl; X₂ is isobutyl; R₄ is t-butyl carbonyl; and P₁ is Boc.) is coupled to C7, C10 di-Cbz 10-deacetylbaccatin III, Formula 2, which also forms coupled product of Formula 22 (discussed above with respect to FIG. 10).

A solution of Formula 23 (5.5 g, 13.47 mmol) in THF (30 mL) was cooled to 0° C. with an ice-water bath and 0.20 mL (1.8 mmol) 99% 4-methylmorpholine and 0.22 mL (1.8 mmol, 0.2 eq) 99% trimethylacetylchloride (pivaloyl chloride) were added. The reaction was stirred at ambient temperature for one hour. To this reaction mixture was then added a solution containing 1.76 g (14.4 mmol, 1.60 eq) 99% 4-DMAP and 7.30 g (8.98 mmol, 1.0 eq) of C7, C10 di-Cbz 10-deacetylbaccatin III, Formula 2, and the reaction was gently heated under reflux for about sixteen (16) hours under an inert atmosphere of nitrogen. After cooling to ambient temperature, the reaction was concentrated to dryness and reconstituted in EtOAc (60 mL). After stirring for about ten minutes, solids were removed by filtration. The filtrate was washed with saturated sodium bicarbonate solution (60 mL), water (60 mL) and brine (60 mL). The organic phase was concentrated to dryness to afford 14.52 g (>100%) crude coupled product, Formula 22. This crude material was dissolved into five volumes of MeOH and added dropwise (slowly) into water (10 volumes) with good stirring. The solids were filtered and dried to constant weight in vacuo at about 45° C. to yield 10.84 g (100%) coupled product, Formula 22, as a white solid; 74.2 AP (230 nm).

Another specific example of coupling as in FIG. 9, Formula 12 (wherein: X is F; X₁ is R₁P₁; X₂ is isobutyl; X₃ is P₂; R₁ is H; P₁ is Boc; and P₂ is BOM) is coupled to C7, C10 di-Cbz 10-deacetylbaccatin III, Formula 2 to yield coupled ester of Formula 13.

Coupling of side chain acid, Formula 12 to alcohol Formula 2:

A solution of acid fluoride (Formula 12) (28.30 gm, 73.81 mmol) in 60 ml of DCM was added dropwise to a stirring solution of alcohol (Formula 2) (50.0 gm, 61.51 mmols) and 4-pyrrolidino pyridine (11.39 gm, 76.89 gm) DCM (250 mL) at room temperature under nitrogen. TLC and LC-MS analysis after 13 hours revealed complete consumption of alcohol (formula 2) with traces of acid fluoride remaining and formation of desired coupled ester (Formula 13). The reaction mixture was diluted with 100 ml of DCM and transferred to a separatory funnel. The DCM layer was washed with water (2×100 ml), brine (100 ml), dried over sodium sulfate (80 gm) and rotostripped to afford a solid (81.80 gm). The crude product was purified on a silica plug, eluting with IPAC. The pure fractions were pooled and the solvents were evaporated to afford the coupled ester (Formula 13) (78.4 gm) as an off white solid after repeated washes with heptanes.

### V. Formation of Side Chains

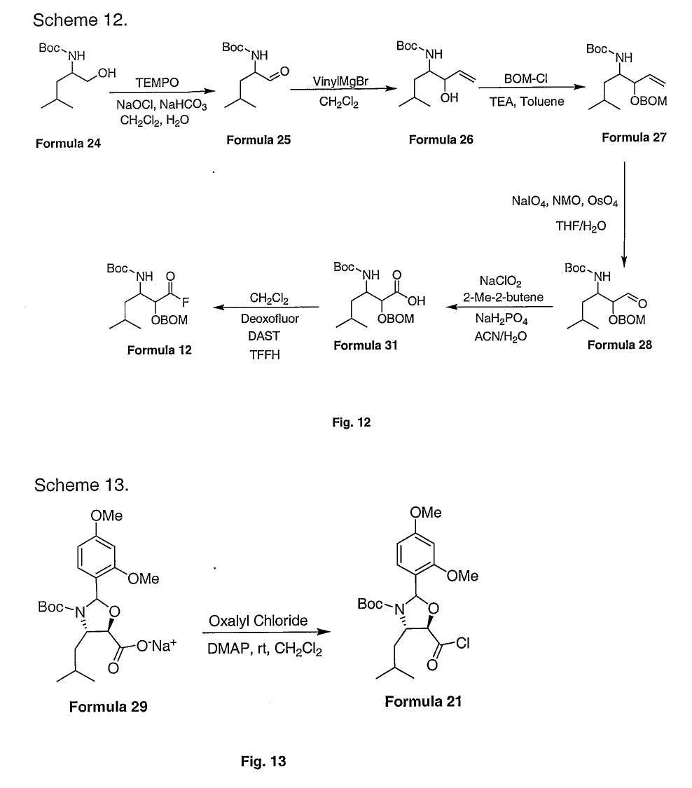

As described above, one of the aspects of the present invention is the novel side chain, generally shown as compound A in Scheme 1a and b (FIG. 1). Thus far, several specific coupling reactions involving various side chains contemplated by the present invention have been described above with reference to FIGS. 6-11 above. Now, the formation of these particular novel side chains can be described with reference first to FIG. 12.

A. Synthesis of Side Chain—Formula 1 and Formula 12

FIG. 12 shows an exemplary process for producing both side chains of Formula 1 and Formula 12.

Formula 26 (prepared as described in J. Org. Chem. 2001, 66, 3330-3337) was converted to formula 27.

Formula 26 (12.1 g, 49.79 mmol) was dissolved in 120 ml of toluene and added to a 250 ml 3-necked flask fitted with a reflux condenser under nitrogen and stirred. TEA (17.34 mL, 124.48 mmol) was added followed by BOM-CI (13.6 g, 87.13 mmol) as the bath was heated to 120° C. After 2.5 hours TLC indicated all of starting material had converted to a faster spot. The reaction was cooled and poured into a separating funnel and diluted with 300 ml EtOAc and washed with 200 ml of 1N HCl. The layers were separated, washed with 300 ml of 5% NaHCO₃, 200 ml brine, dried over Na₂SO₄, filtered and concentrated. Crude product formula 27 used as such in the next step.

Formula 27 (2.0 g, 5.5 mmol) was dissolved in THF (100 mL), chilled to 5° C. and stirred vigorously. In 100 mL water with stirring NaIO₄ (2.35 g, 11.0 mmol) and NMO (1.29 g, 11.0 mmol) were dissolved, which was slowly added to the chilled THF solution. Lastly, OsO₄ (0.035 g, 0.025 eq.) was added. After 6 hours the reaction was complete as indicated by TLC. The THF was stripped under vacuum, sodium thiosulfate solution was added and the mixture was shaken. The resulting aqueous mixture was extracted with EtOAc (3×50 mL). The organic extract was washed with brine, dried over Na₂SO₄, filtered and then concentrated to oil. The aldehyde product formula 28 was used directly in the next step without purification.

Formula 28 (3.44 g, 9.42 mmol) was dissolved in 70 ml of tBuOH and 20 mL water was added and stirring was commenced under nitrogen. Na₂HPO₄ (2.324 g, 16.96 mmol) and 2-methyl 2-butene (18.75 mL, 169.6 mmol) was added and the solution cooled on an ice water bath. Sodium Chlorite (2.03 g, 22.62 mmol) was added over a period of two minutes and the ice bath removed and the solution stirred and allowed to rise to room temperature and stir for one hour. TLC indicated the reaction had gone to completion. 15 mL of Na₂S₂O₃ was added slowly followed by 50 mL of EtOAc. The organic layer was separated and aqueous layer back extracted with 50 mL EtOAc, dried over Na₂SO₄, filtered and then concentrated. Purification by silica gel chromatography gave 2.8 g of acid formula 12.

If desired, side chain of Formula 31 can then be converted to side chain of Formula 12 as shown. By one method, Formula 31 to Formula 12 (acid to acid fluoride) is as follows:

Pyridine (10.7 mL, 131.2 mmol) was added dropwise to a solution of side chain acid (Formula 31) (40.0 g, 104.99 mmol) in dichloromethane (200 ml) at room temperature under an atmosphere of nitrogen. The reaction was then cooled to 10° C. and DAST (Diethylamino sulfur trifluoride) (150.1 mL, 115.48 mmol) was added via a syringe at a rate that the reaction temperature was maintained below −5° C. Stirring was continued for about 2 hours when TLC indicated the reaction was complete. The reaction was quenched at −10° C. by the addition of ice cold water (20 mL).

The mixture was transferred to a separatory funnel and the methylene chloride layer was separated washed with 150 mL of cold water and 100 ml of brine solution. The organic layer was dried over 40 gm of Sodium sulfate and rotostripped to afford the crude acid fluoride as an oil. The crude product was purified on a silica plug, eluting with 25% IPAC in heptanes to afford the clean product Formula 12 as an oil (38.8 g, 96.5%) after the solvents were rotostripped.

For this conversion and for the conversions of other acids to acid fluorides, reagents such as Deoxoflour, cyanuric fluoride and TFFH can also be used in addition to the DAST method shown here. Formula 1 was also synthesized according to the above described fluorination methods.

B. Synthesis of Side Chain—Formula 21 and Formula 23

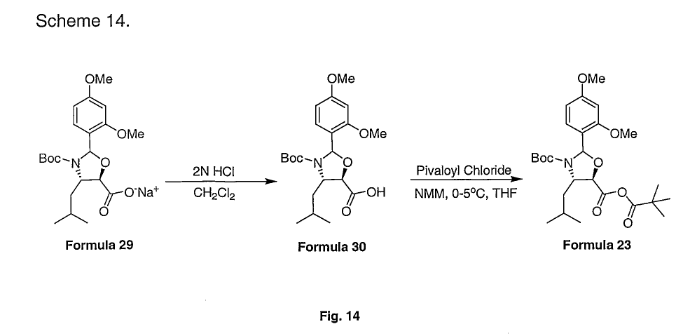

Turning now to FIGS. 13 and 14, side chains, Formula 21 and 23, can both be formed from the side chain, Formula 29. Synthesis of formula 29 is described in WO 01/02407 A2 to Bombardelli et al., which is incorporated herein by reference. As shown in FIG. 13. side chain Formula 29 can be converted to acid chloride side chain, Formula 21. First, a solution was prepared containing 7.96 g (18.4 mmol, 3.0 eq) side chain, Formula 29 and 2.25 g (18.4 mmol) 99% 4-DMAP in anhydrous dichloromethane (80 mL). To this solution, 1.70 mL (19.1 mmol, 3.1 eq) 98% oxalyl chloride (neat) was added at ambient temperature under an inert atmosphere of nitrogen. The resulting mixture was stirred at ambient temperature for about 30 minutes, 98% oxalyl chloride (0.5 mL) was added and the mixture was stirred for an additional 30 minutes. HPLC analysis indicated conversion to acid chloride side chain, Formula 21 was complete (a reaction aliquot was quenched into methanol and analyzed as methyl ester). The mixture was filtered and the solids were washed with anhydrous dichloromethane (30 mL). The filtrate was concentrated under reduced pressure and the oil was further concentrated in vacuo under high vacuum for 25 minutes. The resulting oil was re-dissolved in anhydrous dichloromethane (30 mL) thereby producing a solution containing acid chloride side chain of Formula 21.

Turning to FIG. 14, side chain of Formula 23 can be synthesized from side chain of Formula 29.

A solution containing 55.00 g (127.5 mmol) of side chain, Formula 29, in dichloromethane (550 mL) was washed with cold (0-5° C.) 2N aqueous HCl solution (2×460 mL). The organic phase was washed with 12.5 wt % sodium chloride solution (2×460 mL), dried over anhydrous sodium sulfate, filtered and concentrated in vacuo to constant weight to afford 50.35 g (96.5%) free acid, Formula 30.

To a 0-5° C. solution of 5.51 g (13.5 mmol) free acid Formula 30 in anhydrous THF (50 mL) under an inert atmosphere of nitrogen was added 1.78 mL (16.2 mmol) 99% 4-methylmorpholine and 1.99 mL (16.2 mmol) 99% trimethylacetyl chloride. The progress of the reaction was monitored by HPLC (a reaction aliquot was quenched into MeOH). After one hour, 0.20 mL (1.8 mmol, 0.2 eq) 99% 4-methylmorpholine and 0.22 mL (1.8 mmol, 0.2 eq) 99% trimethylacetyl chloride were added. After an additional 30 minutes at 0-5° C., the conversion to the mixed anhydride side chain, Formula 23 was complete.

1. A method for use in producing taxanes, taxane analogs, and derivatives thereof, comprising the step of reacting a first compound of the general formula: 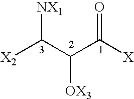
with a second compound of the general structure: 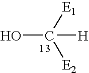
to give a third compound of the general formula: 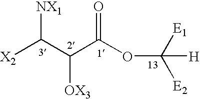
wherein:
X is a halogen or OR₄;
X₁ is either R₁R₂; R₁P₁; R₂P₁; or P₁P₁
X₂ is a substituted or unsubstituted: alkyl, alkenyl, aryl, aralkyl, or acyl;
X₃ is either R₁; R₂; or P₂;
R₁ and R₂ are independently H or substituted or unsubstituted: alkyl alkenyl, aryl, aralkyl, or acyl;
R₄ is a substituted or unsubstituted: alkenyl, aryl, aralkyl, acyl, alkoxy carbonyl or aryloxy carbonyl, aroyl or alkali metal;
P₁ is an amine protecting group;
P₂ is a hydroxyl protecting group; and
E₁, E₂ and the carbon to which they are attached define a tetracyclic taxane nucleus.

2. A method according to claim 1 wherein the second compound has a structure 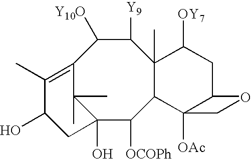
Y₇ is R₇; P₃; or Z₇;
Y₉ is H; hydroxyl; a ketone; OR₉; P₄; or Z₉;
Y₁₀ is R₁₀; P₅; or Z₁₀;
Z₇ is P₃ and together with Y₉ forms a cyclic structure when Y₉ is P₄;
Z₉ is either:
P₄ and together with Y₇ forms a cyclic structure when Y₇ is P₃; or P₅ and together with Y₁₀ forms a cyclic structure when Y₁₀ is P₄;
Z₁₀ is P₅ and together with Y₉ forms a cyclic structure when Y₉ is P₄;
R₇ is H, substituted or unsubstituted: alkyl, alkenyl, aryl, aralkyl, or acyl;
R₉ is a substituted or unsubstituted: alkyl, alkenyl, aryl, aralkyl, or acyl;
R₁₀ is H, substituted or unsubstituted: alkyl, alkenyl, aryl, aralkyl, or acyl;
P₃ is a hydroxyl protecting group;
P₄ is a hydroxyl protecting group; and
P₅ is a hydroxyl protecting group.

3. A method according to claim 2 wherein X is a halogen; X₁ is R₁P₁; X₂ is Ph; X₃ is P₂; Y₇ is P₃; Y₉ is a ketone; Y₁₀ is P₅; R₁ is H; P₁ is Boc; P₂ is BOM; P₃ is Cbz; and P₅ is Cbz.

4. A method according to claim 2 wherein X is fluorine; X₁ is R₁P₁; X₂ is Ph; X₃ is P₂; Y₇ is P₃; Y₉ is a ketone; Y₁₀ is P₅; R₁ is H; P₁ is Cbz; P₂ is BOM; P₃ is Cbz; and P₅ is Cbz.

5. A method according to claim 2 wherein X is OR₄; X₁ is R₁P₁; X₂ is isobutyl; X₃ is P₂; Y₇ is P₃; Y₉ is a ketone; Y₁₀ is P₅; R₁ is H; P₁ is Boc; P₂ is BOM; P₃ is Cbz; and P₅ is Cbz.

6. A method according to claim 2 wherein X is a halogen; X₂ is isobutyl; Y₇ is P₃; Y₉ is a ketone; Y₁₀ is P₅; R₁ and R₂ are independently H or substituted or unsubstituted: alkyl, alkenyl, aryl, aralkyl, or acyl; R₃ is H; P₁ is Boc; P₂ is BOM; P₃ is Cbz; and P₅ is Cbz.

7. A method according to claim 1 wherein said third compound has the formula:

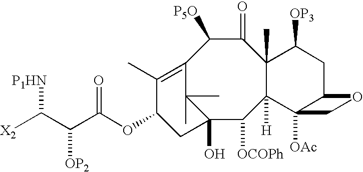

8. A method according to claim 7 wherein said third compound is converted to docetaxel, paclitaxel, or a 7,9-acetal linked analog.

9. A method according to claim 7 wherein said third compound is deprotected by substituting hydrogen for P₁, P₂, P₃ and P₅ to form a fourth compound having the formula:

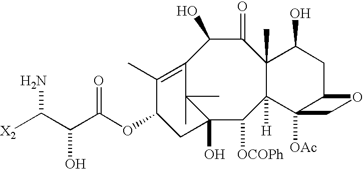

10. A method according to claim 7 wherein said third compound is deprotected by substituting hydrogen for P₃, and P₅ to form a fourth compound having the formula:

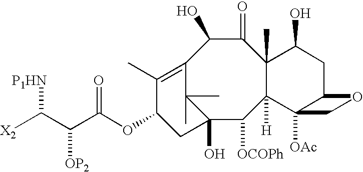

11. A method according to claim 10 wherein said fourth compound is selectively acylated at the C-10 position to form a fifth compound having the formula:

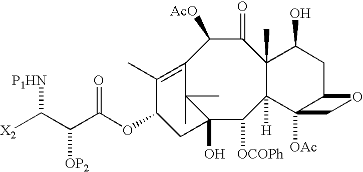

12. A method according to claim 11 wherein said fifth compound is converted to paclitaxel.

13. A method according to claim 7 wherein said third compound is oxidized to form a fourth compound of the formula:

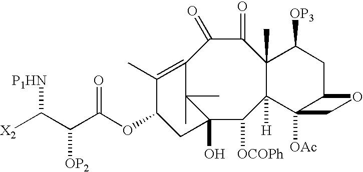

14. A method according to claim 13 wherein said fourth compound is reduced to form a fifth compound of the formula:

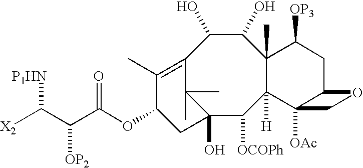

15. A method according to claim 14 wherein said fifth compound is acylated at the C-10 position to form a sixth compound of the formula:

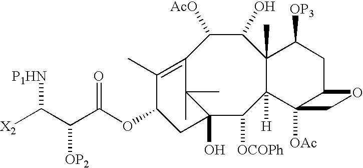

16. A method according to claim 15 wherein said sixth compound deprotected by substituting hydrogen for P₃ thereby to form a seventh compound of the formula:

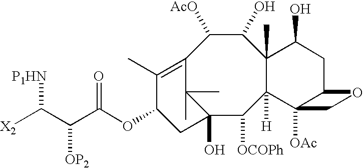

17. A method according to claim 16 wherein said seventh compound is converted to an eighth compound of the formula: 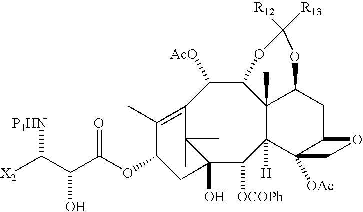
wherein R₁₂ and R₁₃ are independently H; substituted or unsubstituted: alkyl; alkenyl; aryl; aralkyl; or acyl.

18. A method according to claim 17 wherein R₁₂ and R₁₃ are each independently selected from the group consisting of:

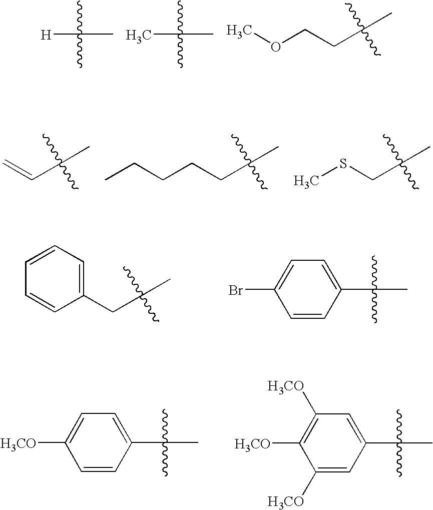

19. A method according to claim 1 wherein said third compound has the formula

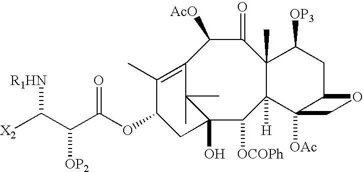

20. A method according to claim 19 wherein said third compound is converted to paclitaxel.

21. A method according to claim 1 wherein the first compound is a cyclic structure wherein the C-3 Nitrogen and the C-2 Oxygen are linked by a common protecting group that includes R₁ and R₂ and that has the formula: 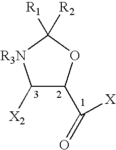
such that the third compound is a cyclic structure having the formula: 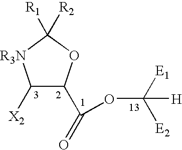
wherein R₃ is either H or P₁.

22. A method according to claim 21 wherein the second compound has a structure 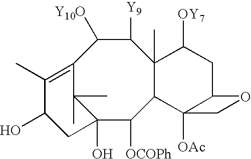
Y₇ is R₇; P₃; or Z₇;
Y₉ is H; hydroxyl; a ketone; OR₉; P₄; or Z₉;
Y₁₀ is R₁₀; P₅; or Z₁₀;
Z₇ is P₃ and together with Y₉ forms a cyclic structure when Y₉ is P₄;
Z₉ is either:
P₄ and together with Y7 forms a cyclic structure when Y₇ is P₃; or P₅ and together with Y₁₀ forms a cyclic structure when Y₁₀ is P₄;
Z₁₀ wherein P₅ forms a cyclic structure with P₄;
R₇ is H, substituted or unsubstituted: alkyl, alkenyl, aryl, aralkyl, or acyl;
R₉ is a substituted or unsubstituted: alkyl, alkenyl, aryl, aralkyl, or acyl;
R₁₀ is H, substituted or unsubstituted: alkyl, alkenyl, aryl, aralkyl, or acyl;
P₃ is a hydroxyl protecting group;
P₄ is a hydroxyl protecting group; and
P₅ is a hydroxyl protecting group.

23. A method according to claim 22 wherein X is a halogen; X₂ is Ph; Y₇ is P₃; Y₉ is a ketone; Y₁₀ is P₅; R₁ is H; P₁ is Boc; P₂ is BOM; P₃ is Cbz; and P₅ is Cbz.

24. A method according to claim 21 wherein X is fluorine; X₁ is R₁P₁; X₂ is Ph; X₃ is P₂; Y₇ is P₃; Y₉ is a ketone; Y₁₀ is P₅; R₁ is H; P₁ is Cbz; P₂ is BOM; P₃ is Cbz; and P₅ is Cbz.

25. A method according to claim 22 wherein X is OR₄; X₁ is R₁P₁; X₂ is isobutyl; X₃ is P₂; Y₇ is P₃; Y₉ is a ketone; Y₁₀ is P₅; R₁ is H; P₁ is Boc; P₂ is BOM; P₃ is Cbz; and P₅ is Cbz.

26. A method according to claim 22 wherein X is a halogen; X₂ is isobutyl; Y₇ is P₃; Y₉ is a ketone; Y₁₀ is P₅; R₁ and R₂ are independently H or substituted or unsubstituted: alkyl, alkenyl, aryl, aralkyl, or acyl; R₃ is H; P₁ is Boc; P₂ is BOM; P₃ is Cbz; and P₅ is Cbz.

27. A chemical compound having the formula: 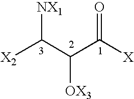
X is a halogen or OR₄;
X₁ is either R₁R₂; R₁P₁; R₂P₁; or P₁P₁
X₂ is a substituted or unsubstituted: alkyl, alkenyl, aryl, aralkyl, or acyl;
X₃ is either R₁; R₂; or P₂;
R₁ and R₂ are independently H or substituted or unsubstituted: alkyl, alkenyl, aryl, aralkyl, or acyl;
R₄ is a substituted or unsubstituted: alkenyl, aryl, aralkyl, acyl, alkoxy carbonyl, aroyl or aryloxy carbonyl;
P₁ is an amine protecting group;
P₂ is a hydroxyl protecting group.

28. A chemical compound according to claim 27 wherein X is selected from the group consisting of chlorine, bromine, fluorine, and iodine.

29. A chemical compound according to claim 27 is a cyclic structure wherein the C-3 Nitrogen and the C-2 Oxygen are linked by a common protecting group that includes R₁ and R₂ and that has the formula: 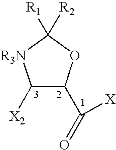
wherein R₃ is either H or P₁.

30. A chemical compound according to claim 29 wherein X is selected from the group consisting of chlorine, bromine, fluorine, and iodine.

31. A chemical compound according to claim 29 wherein X is chlorine; X₂ is isobutyl; R₁ and R₂ are independently H, or substituted or unsubstituted: alkyl, alkenyl, aryl, aralkyl or acyl; R₃ is P₁; and P₁ is Boc.

32. A chemical compound according to claim 31 having the structural formula:

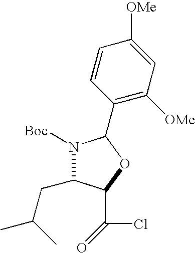

33. A chemical compound according to claim 29 wherein X is R₄; X₂ is isobutyl; R₁ and R₂ are independently H, or substituted or unsubstituted: alkyl, alkenyl, aryl, aralkyl or acyl; R₃ is P₁, R₄ is trimethylacetyl; and P₁ is Boc.

34. A chemical compound according to claim 33 having the structural formula:

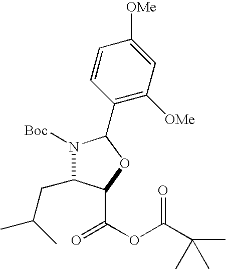

35. A chemical compound according to claim 27 wherein X is OR₁₁; X₁ is R₁P₁; X₂ is isobutyl; X₃ is P₂; R₁ is H; R₁, is H; P₁ is Boc; and P₂ is BOM.

36. A chemical compound according to claim 35 having the structural formula:

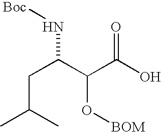

37. A chemical compound according to claim 27 wherein X is fluorine; X₁ is R₁P₁; X₂ is isobutyl; X₃ is P₂; R₁ is H; P₁ is Boc; and P₂ is BOM.

38. A chemical compound according to claim 37 having the structural formula:

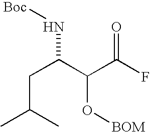

39. A chemical compound according to claim 27 wherein X is OR₁₁; X₁ is R₁P₁; X₂ is Ph; X₃ is P₂; R₁ is H; R₁₁ is H; P₁ is Cbz; and P₂ is BOM.

40. A chemical compound according to claim 39 having the structural formula:

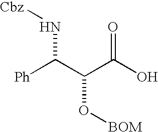

41. A chemical compound according to claim 27 wherein X is fluorine; X₁ is R₁P₁; X₂ is Ph; X₃ is P₂; R₁ is H; P₁ is Cbz; and P₂ is BOM.

42. A chemical compound according to claim 41 having the structural formula:

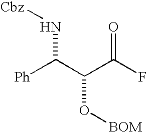

43. A compound according to claim 29 wherein X is R₄; X₂ is aryl, substituted aryl, heteroaryl or substituted heteroaryl; R₁ and R₂ are methyl; R₃ is P₁, R₄ is trimethylacetyl; and P₁ is Boc.

44. A compound of the formula: 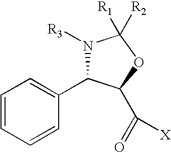
X is a halogen or OR₄;
R₁ and R₂ are independently H or substituted or unsubstituted: alkyl, alkenyl, aryl, aralkyl, or acyl;
R₃ is an amine protecting group;
R₄ is a substituted or unsubstituted: alkenyl, aryl, aralkyl, acyl, alkoxy carbonyl, aroyl or aryloxy carbonyl.

45. A compound of the formula [chemical structure]
